(19) United States
(12) Patent Application Publication
(10) Pub. No.: US 2015/0104881 A1
(43) Pub. Date: Apr. 16, 2015

(54) SGLT2 AS BIOMARKER FOR DIAGNOSING CHRONIC KIDNEY DISEASE AND MONITORING THE DISEASE STATUS AFTER TREATMENT

(71) Applicant: National Defense Medical Center, Taipei (TW)

(72) Inventors: Ann CHEN, Taipei (TW); Shuk-Man KA, Taipei (TW)

(21) Appl. No.: 14/512,725

(22) Filed: Oct. 13, 2014

(30) Foreign Application Priority Data
Oct. 11, 2013 (TW) 102136857

(51) Int. Cl. G01N 33/68

(52) U.S. Cl. CPC G01N 33/6893; G01N 2800/347
USPC 436/501

(57) ABSTRACT

The present invention relates to a method for diagnosis of a chronic kidney disease in a subject by detecting the expression level of a sodium-glucose linked transporter 2 (SGLT2) protein in urine samples. A method for monitoring progression of a chronic kidney disease in a patient is also provided.

### RELATED APPLICATIONS

This application claims the benefit of Taiwanese Patent Application No. 102136857, filed on Oct. 11, 2013, the entire content of which is incorporated herein by reference.

### TECHNOLOGY FIELD

The present invention discloses that sodium-glucose linked transporter 2 (SGLT2) can be used as a biomarker for diagnosing a chronic kidney disease and monitoring the disease status.

### BACKGROUND OF THE INVENTION

Chronic kidney diseases (CKDs) are diseases where the kidney was damaged by either diseases or drugs so that renal function was impaired and cannot recover to the original normal state. Typical chronic kidney diseases include focal segmental glomerulosclerosis (FSGS) and IgA nephropathy (IgAN). If not diagnosed and treated in time, CKDs can easily develop into chronic renal failure, where patients eventually lose their renal function and enter into end-stage renal diseases.

Chronic renal failure usually shows no symptom for a very long time, but cannot be reversed once it has occurred. Therefore, if chronic kidney diseases can be diagnosed in an early stage and interrupted by preventive treatment as soon as possible, the occurrence of chronic renal failure can be effectively avoided, or deterioration of the same can be delayed. However, to date there in no easy diagnostic reagents for chronic kidney diseases, and they have to be determined by the results of clinical symptoms in combination with components in the serum and urine and tissue biopsy. Among them, although detection of serum creatinine and urine albumin has been extensively used in the diagnosis and follow-up, it has a number of disadvantages such as insufficient sensitivity and specificity and difficulties in evaluating early symptoms. As for renal biopsy, it is an invasive test which is dangerous for patients as it may lead to some serious complications of haemorrhage and this lowers patients' willingness to be tested, especially in an early stage when no severe symptoms occur, and thus delays detection of the disease. In addition, once diagnosed as having a chronic kidney disease, the patient usually requires long-term treatment. During the period, the patient's disease status must be continuously monitored in order to confirm the suitability of the treatment and to avoid disease progression. An invasive test also largely reduces patients' willingness for continuous monitoring.

Therefore, there is a need in this field to develop new techniques for diagnosing chronic kidney diseases and monitoring post-treatment disease status, especially those that employ non-invasive methods so as to increase acceptance among patients to achieve the purpose of early diagnosis and continuous monitoring of the disease status.

### BRIEF SUMMARY OF THE INVENTION

In the present invention, it is unexpectedly found that that the amount of a sodium-glucose linked transporter 2 (SGLT2) protein in urine samples from animals with a chronic kidney disease is significantly elevated, when compared to those from normal animals or diseased animals after treatment. Therefore, sodium-glucose linked transporter 2 (SGLT2) can be used as a biomarker, via urine tests, for diagnosing a chronic kidney disease and also monitoring the disease status.

In one aspect, the present invention provides a method for diagnosing a chronic kidney disease in a subject, comprising

(a) obtaining a urine sample from the subject;

(b) determining a level of a sodium-glucose linked transporter 2 (SGLT2) protein in the urine sample; and

(c) identifying the subject as having or at risk for the chronic kidney disease, if the level of the SGLT2 protein in the urine sample is increased relative to the level of the SGLT2 protein in urine from a control.

In another aspect, the present invention provides a method for monitoring progression of a chronic kidney disease (CKD), comprising

(a) providing a first urine sample from a patient with CKD at a first time point;

(b) providing a second urine sample from the patient at a second time point; which is later than the first time point;

(c) measuring levels of a sodium-glucose linked transporter 2 (SGLT2) protein in the first and second urine samples by an immunoassay; and

(d) determining progression of the chronic kidney disease in the patient based on the levels of the SGLT2 protein in the first and second urine samples, wherein an increased level of the SGLT2 protein in the second urine sample relative to that in the first urine sample is indicative of progression of the chronic kidney disease in the patient

Specifically, the urine samples are obtained in a non-invasive way.

In certain embodiments, the chronic kidney disease is focal segmental glomerulosclerosis (FSGS) or IgA nephropathy (IgAN).

In some embodiments, the SGLT2 level in urine samples is measured by an immunoassay. Examples of an immunoassay include but not limited to western blotting or enzyme-linked immunosorbent assay (ELISA).

Other characteristics of the present invention will be clearly presented through the following detailed description, multiple embodiments, and claims.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

For the purpose of illustrating the invention, there are shown in the drawings embodiments which are presently preferred. It should be understood, however, that the invention is not limited to the illustrated preferred embodiments. In the drawings:

FIG. 1 shows the kidney histopathological evaluation by hematoxylin and eosin (H&E) staining in normal mice and FSGS or IgAN mice, without or with treatment osthole, wherein (A) refers to the FSGS mouse model and (B) refers to the IgAN mouse model.

FIG. 2 shows the level of a SGLT2 protein in urine samples from normal mice and FSGS or IgAN mice, without or with treatment osthole, wherein (A) refers to the results of western blotting, (B) refers to the results of the SGLT2 levels after calibration with urine creatinine concentrations (FSGS model), and (C) refers to the results of the SGLT2 levels after calibration with urine creatinine concentrations (IgAN model). * p<0.05, ** p<0.01 as compared to the disease group.

### DETAILED DESCRIPTION OF THE INVENTION

Unless defined otherwise, all technical and scientific terms used herein have the same meanings as is commonly understood by one of skill in the art to which this invention belongs. If a conflict appears, one should base on this document, including the definitions therein.

As used herein, the articles “a” and “an” refer to one or more than one (i.e., at least one) of the grammatical object of the article. By way of example, “an element” means one element or more than one element.

The term “expression” used herein refers to the realization of genetic information encoded in the genes to produce genetic products, such as unspliced RNAs, mRNAs, splice variant mRNAs, polypeptides or proteins, post-translationally modified polypeptides, and splice variant polypeptides. In particular, in the present invention, the term “expression” can refer to production of polypeptides or proteins from a gene.

The term “expression level” or “level” used herein refers to the amount of genetic products from the expression of a specific gene, which can be determined by any appropriate method known in the art. In one embodiment of the present invention, the term “expression level” refers to the amount of polypeptides or proteins expression from a specific gene.

The terms “polypeptide” and “protein” used herein can be used interchangeably, referring to the polymeric form of amino acids of any length, which includes coded and non-coded amino acids, chemically or biochemically modified or derivatized amino acids, or polypeptides with a modified peptide backbone.

The term “antibody” used herein refers to an immunoglobulin that can bind to an antigen. The term “antibody” used herein includes whole antibodies and any antibody fragments (such as F(ab′).sub.2, Fab′, Fab, and Fv) that can bind to an epitope, antigen, or antibody fragment of interest. The antibodies used in the present invention exhibit immunoreaction or immunospecificity to designated proteins and therefore can specifically and selectively bind to the same. For example, the SGLT2 expression level in a sample can be determined with SGLT2-specific antibodies. Antibodies to a protein of interest preferably have immunospecificity, meaning that they can recognize an interspecies homolog but would not exhibit substantial cross-reaction to the relevant material. The term “antibody” used herein includes all types of antibodies (such as monoclonal and polyclonal antibodies.)

The terms “subject,” “individual,” and “patient” used herein can be used interchangeably, and refer to any mammal subject in need of diagnosis, treatment, or medical attention, specifically human. Other subjects may include cows, dogs, cats, guinea pigs, rabbits, rats, mice, and horses.

The term “diagnosis” used herein refers to the determination or evaluation of the probability of an individual to suffer from a certain disease, disorder, or dysfunction. Persons skilled in the art often perform diagnosis based on one or more diagnostic indices (i.e., markers), wherein the existence, absence, or amount of such diagnostic indices may be an indicator of whether said disease, disorder or dysfunction exists. It should be understood in the art that the determination or evaluation here does not require 100% accuracy for all individuals, but identification of a population with statistical significance would be enough. The aforementioned determination or evaluation can be performed using various known statistical methods in the art. Generally, when a biomarker shows a positive correlation with the existence or development of a disease, said biomarker can be used as a diagnostic tool for said disease.

The term “treatment” used herein can refer to the application or administration of a composition comprising one or more active ingredients to an individual suffering from a disease or symptoms of said disease, or having a tendency to contract said disease, for the purpose of curing, healing, alleviating, ameliorating, changing, correcting, improving, reforming, or influencing said disease, symptoms of said disease, disabilities induced by said disease, or tendency to contract said disease.

As known in the art, focal segmental glomerulosclerosis (FSGS), a typical example of chronic kidney diseases, is a kidney disease where scaring (sclerosis) occurs in a portion of some (focal) but not all glomeruli, which can be identified by a biopsy of renal tissue. FSGS shows pathological symptoms including glomerular epithelial hyperplasia lesions (EPHLs), a key histopathology index of progression of FSGS, and also peri-glomerular inflammation and glomerular hyalinosis or sclerosis. FSGS also shows characteristics of renal dysfunction symptoms such as severe proteinuria, hypertension, hypoalbuminemia and hematuria etc. (Cattran D C, Rao P. American Journal of Kidney Disease, 21(3):344-9, 1998; Chun M J et al., Journal of the American Society of Nephrology, 15:2169, 2004; Rydel J J et al., American Journal of Kidney Disease, 25(4):534-42, 1995). As the progression of the disease, the symptoms include thickening of the glomerular basement membrane, increasing of glomerular extracellular matrix, appearance of glass-like deposits in blood vessels, and then formation of scar tissues composed mainly of collagen, accompanied by accumulation of foam cells on the capillary wall, capillary collapse, hyperplasia and hypertrophy of viceral epithelial cells, and podocyte fusion; the sclerotic portion expands gradually with the progression of the disease (D'Agati V D. Curr Opin Nephrol Hypertens 17(3):271-81, 2008; Hodgin J B et al., American Journal of Clinical Pathology, 177(4):1674-86, 2010; and Thomas D B., The Archives of Pathology and Laboratory Medicine. 133(2):217-23, 2009).

As known in the art, “IgA nephropathy (IgAN)” is a kidney disease characterized by IgA1 deposits within the kidney, which is also a typical example of chronic kidney diseases. The most common histopathologic alteration associated with IgAN is focal or diffuse expansion of mesangial regions with proliferative cells and extracellular matrix. In addition, a wide variety of lesions identified by light microscopy may be seen in patients with more severe lesions, including diffuse endocapillary proliferation, segmental sclerosis, segmental necrosis, and cellular crescent formation. Several factors have been confirmed to highly correlated with an unfavorable prognosis of IgA nephropathy including hematuria, proteinuria, moderate hypercellularity, glomerulosclerosis, tubulointerstitial inflammation, and a diffuse glomerular co-deposition of IgG and/or IgM as well as complement components 3 (C3).

The term “sodium-glucose linked transporter 2 (SGLT2)” described herein is a member of the sodium-glucose transport protein family, which is an important transport protein responsible for reabsorption of glucose in the kidney. It is known that inhibition of expression of SGLT2 can effectively reduce the blood glucose concentration, and thus SGLT2 inhibitors can be used in treating Type II diabetes. The nucleotide sequence of the SGLT2 gene and the amino acid sequence of its expressed genetic product are well known in the art.

The present invention discloses for the first time that a SGLT2 expression level in urine samples of a subject is highly associated with occurrence and development of a chronic kidney disease, particularly FSGS or IgAN.

Therefore, in one aspect, SGLT2 can be used as a diagnostic marker for chronic kidney disease. According to the present invention, it is found that a SGLT2 expression level in urine samples from a normal animal is undetected or very low; however, a SGLT2 expression level in urine samples from a FSGS or IgAN animal is relatively high. Therefore, one can determine whether a subject suffers from or is at risk for a chronic kidney disease based on a SGLT2 expression level in urine samples from the subject.

In particular, the present invention provides a method for diagnosing a chronic kidney disease in a subject, comprising

(a) obtaining a urine sample from the subject;

(b) determining a level of a sodium-glucose linked transporter 2 (SGLT2) protein in the urine sample; and

(c) identifying the subject as having or at risk for the chronic kidney disease, if the level of the SGLT2 protein in the urine sample is increased relative to the level of the SGLT2 protein in urine from a control.

In particular, the chronic kidney disease is FSGS or IgA nephropathy.

The term a “normal” subject or a “control” described herein can be used interchangeably, which refers to a healthy normal subject who is not suffering from a chronic kidney disease e.g. FSGS or IgAN. They may refer to a single normal subject or a population of a group of normal subjects.

The term a “higher” “increased” or “elevated” expression level as described herein can mean that such level is higher than a control level measured in urine samples from a normal subject. In some embodiments, a higher level can be higher than a control level by more than 10%, 20%, 30%, 40%, 50%, 60%, 70%, 80%, 90% or 100%.

In some embodiments, a subject to be analyzed by the method of the invention can be a subject who already exhibits one or more CKD symptoms. In some embodiments, the diagnostic method of the invention can be used as a routine screening assay on a subject with a generally normal condition for determining risk for CKD.

To perform the methods of the invention, a urine sample is obtained from a subject in need and tested for the SGLT2 level. If an elevated level of the SGLT2 protein in the urine sample is detected, the subject is identified as having or at risk for CKD. In some embodiments, a SGLT2 level in a urine sample obtained from a candidate subject can be compared with a control level to determine if the candidate subject has an elevated SGLT2 level in the urine sample (e.g. higher than the control level by about 10% or more) and determine if he/she has or is at risk for CKD accordingly.

The presence or level of a SGLT2 protein in urine samples can be determined by an immunoassay. Examples of immunoassays include, but are not limited to, western blot, enzyme-linked immunosorbent assay (ELISA), radioimmunoassay (RIA), radioimmunoprecipitation assay (RIPA), immunofluorescence assay (IFA) and electrochemiluminescence (ECL).

To perform an immunoassay, antibodies as used may be polyclonal or monoclonal. Polyclonal antibodies directed against a particular protein are prepared by injection of a suitable laboratory animal with an effective amount of the peptide or antigenic component, collecting serum from the animal, and isolating specific sera by any of known immunoadsorbent techniques. Animals which can readily be used for producing polyclonal antibodies as used in the present invention include chicken, mice, rabbits, rats, goats, horses and the like. In general, use of monoclonal antibodies in detection assays is preferred because of large quantities of antibodies and similar reactivity. Preparation of hybridoma cell lines for producing monoclonal antibodies can be done by fusing an immortal cell line and antibody producing lymphocytes, which can be performed by techniques known in the art.

After a candidate subject has been identified as having or at risk for CKD, such subject can be subjected to a further test (such as tissue biopsy by collecting small pieces of tissue, usually through a needle, for examination with a microscope) to confirm occurrence or status of the disease.

In some embodiments, the method of the invention can further comprise treating the subject with a therapy to treat CKD. Some existing therapeutic methods or medicaments are available, such as plasmapheresis or protein adsorption, or pharmaceutical treatment, including but not limited to corticosteroids (such as prednisolone), non-steriodal anti-inflammatory drugs (NSAIDs), cytotoxic drugs (such as cyclophosphamide, chlorambucil, and azathioprine), immunosuppressants (such as cyclosporine and Mycophenolate Mofetil), and vasodilators (such as angiotensin-converting-enzyme inhibitors (ACE inhibitors)).

In another aspect, SGLT2 can be used as a marker for monitoring progression of CKD.

In particular, the present invention provides a method for monitoring progression of a chronic kidney disease (CKD), comprising

(a) providing a first urine sample from a patient with CKD at a first time point;

(b) providing a second urine sample from the patient at a second time point, which is later than the first time point;

(c) measuring levels of a sodium-glucose linked transporter 2 (SGLT2) protein in the first and second urine samples by an immunoassay; and

(d) determining progression of the chronic kidney disease in the patient based on the levels of the SGLT2 protein in the first and second urine samples, wherein an increased level of the SGLT2 protein in the second urine sample relative to that in the first urine sample is indicative of progression of the chronic kidney disease in the patient.

According to the present invention, a SGLT2 level in urine samples can be measured to determine disease progression. Specifically, at least two or multiple urine samples can be obtained from a candidate patient at different time points and the levels of a SGLT2 protein can be measured as described herein. If a trend of increase in the SGLT2 protein level is observed over time (for example, the SGLT2 level in a later obtained sample is higher than that in an earlier obtained sample), it is indicative of CKD progression in the patient.

In some embodiments, the method of the invention can be used to assay the efficacy of a therapy to treat CKD. For example, at least two urine samples can be obtained from a CKD patient who undergoes a therapy to treat CKD over the course of the therapy. By measuring the SGLT2 levels in these urine samples, if a trend of decrease in the SGLT2 level is observed over the course of the therapy (for example, the SGLT2 level in a later obtained sample is lower than the SGLT2 level in an earlier obtained sample), it indicates that the therapy is effective on the CKD patient. On the contrary, if the SGLT2 level keeps the same (at a high level) or even increases over the course of the therapy, it indicates that the therapy might not be effective on the patient. Based on the results, the therapy can be adjusted property such as increasing drug dosage or administration frequency or change to an alternative therapy.

The present invention will now be described more specifically with reference to the following embodiments, which are provided for the purpose of demonstration rather than limitation.

1. Materials and Methods

1.1 Mouse FSGS Model

A progressive type of mouse FSGS model was used in this study. Experiments were performed on 8-week-old female BALB/c mice. Mice were intravenously injected via tail vein with a single dose of adriamycin (0.25 mg/kg body weight) to induce FSGS as described previously (Tsai P Y et al. (2011) Free Radic Biol Med 50: 1503-1516; Shui H A et al. (2007) Transl Res 150: 216-222). Starting from 3 days before adriamycin injection, mice were given daily osthole by intraperitoneal (i.p.) injection (30 mg/kg) until the day of sacrifice (the osthole treatment group). Age- and sex-matched BALB/c mice injected intravenously with normal saline were used as normal controls, while FSGS mice (without osthole treatment) were used as disease controls.

Table 1 shows the treatment of each group of mice.

TABLE 1

| | Group | | | | | | |
|---|---|---|---|---|---|---|---|
| | Normal | Disease | | | Osthole | | |
| | Control Group | Control Group | | | Treatment Group | | |
| | Number | | | | | | |
| | | N = 6 | | | N = 6 | | |
| | N = 2 | Name | Method | Dosage | Name | Method | Dosage |
| FSGS | | Adria- | IV | 0.25 | Adria- | IV | 0.25 |
| Induction | | mycin | | mg/kg/ | mycin | | mg/kg/ |
| | | | | single | | | single |
| | | | | dose | | | dose |

| | | | |
|---|---|---|---|
| Medical | Osthole | IP | 30 |
| Treatment | | | mg/kg |
| | | | daily |

IV: Intravenous Injection;
IP: Intraperitoneal Injection

Osthole used in this experiment was purified from the seeds of Cnidium monnieri and confirmed to have the chemical name 7-methoxy-8-(3-methylbut-2-enyl)-2-chromanone and the chemical formula C₁₅H₁₆O₃.

All animal experiments were performed with the ethical approval of the Institutional Animal Care and Use Committee of The National Defense Medical Center, Taiwan and according to the ethical rules in the NIH Guide for the Care and Use of Laboratory Animals.

1.2 Mouse IgAN Model

A progressive type of mouse IgAN model (Prg-IgAN) was used in this study. Prg-IgAN was induced in B-cell-deficient (BCD) mice by daily injection of purified IgA anti-phosphorylcholine and pneumococcal C-polysaccharide (PnC) as described previously (Kidney Int 2006; 70: 283-297). Briefly, male C57BL/6 mice were intraperitoneal injected with anti-phosphorylcholine IgA antibody (50 μg/day) and intravenously injected via the tail vein with Pneumococcal C-polysaccharide (PnC) as an antigen (50 μg/day) to induce IgAN. Starting from 3 days before induction of IgAN, mice were given daily osthole by intraperitoneal (i.p.) injection (30 mg/kg) until the day of sacrifice (the osthole treatment group). Age- and sex-matched C57BL/6 mice injected intravenously via tail vein with normal saline were used as normal controls, while IgAN mice (without osthole treatment) were used as disease controls.

TABLE 2
shows the treatment of each group of mice.

| | Group | | | | | | |
|---|---|---|---|---|---|---|---|
| | Normal | Disease | | | Osthole | | |
| | Control Group | Control Group | | | Treatment Group | | |
| | Number | | | | | | |
| | | N = 4 | | | N = 4 | | |
| | N = 2 | Name | Method | Dosage | Name | Method | Dosage |
| IgAN | | IgA | IP | 50 | IgA | IP | 50 |
| Induction | | anti-PC | | μl/ | anti-PC | | μl/ |
| | | | | day | | | day |
| | | PnC Ag | IV | 50 | PnC Ag | IV | 50 |
| | | | | μg/ | | | μg/ |
| | | | | day | | | day |

| | | | |
|---|---|---|---|
| Medical | Osthole | IP | 30 |
| Treatment | | | mg/kg |
| | | | daily |

IV: Intravenous Injection;
IP: Intraperitoneal Injection

1.3 Analysis of Urine Protein and Renal Function

Urine samples were collected in metabolic cages on days 3, 7, 14, and 21. Mice were killed at day 28 post disease induction. Organs (including spleen and renal cortical tissue) and blood samples were collected and stored appropriately until analyses were executed. The concentration of urine protein was determined using BCA kits (Pierce, Rockford, Ill.) as described previously (Shui H A et al., Nephrol Dial Transplant 21: 1794-1802) and normalized to urine creatinine (Cr) levels measured using kits (Wako Pure Chemical Industries, Osaka, Japan), as described previously. Serum levels of blood urea nitrogen (BUN) and Cr were determined using BUN kits and Cr kits (both from Fuji Dry-Chem Slide, Fuji Film Medical, Tokyo, Japan), as described previously (Ka et al. (2012) Diabetologia 55: 509-519).

1.4 Pathologic Evaluation

Renal tissues were formalin-fixed, embedded in paraffin, and sections (4 μm) prepared and stained with hematoxylin and eosin (H&E) for renal histopathology as described previously (Tsai et al. (2011) Free Radic Biol Med 50: 1503-1516; J Am Soc Nephrol 2007; 18: 1777-1788).

2. Results

2.1 Establishment of FSGS Model

Mice were intravenously injected the via tail vein with a single dose of adriamycin (0.25 mg/kg body weight) to induce FSGS as described previously (Tsai P Y et al. (2011) Free Radic Biol Med 50: 1503-1516; Shui H A et al. (2007) Transl Res 150: 216-222).

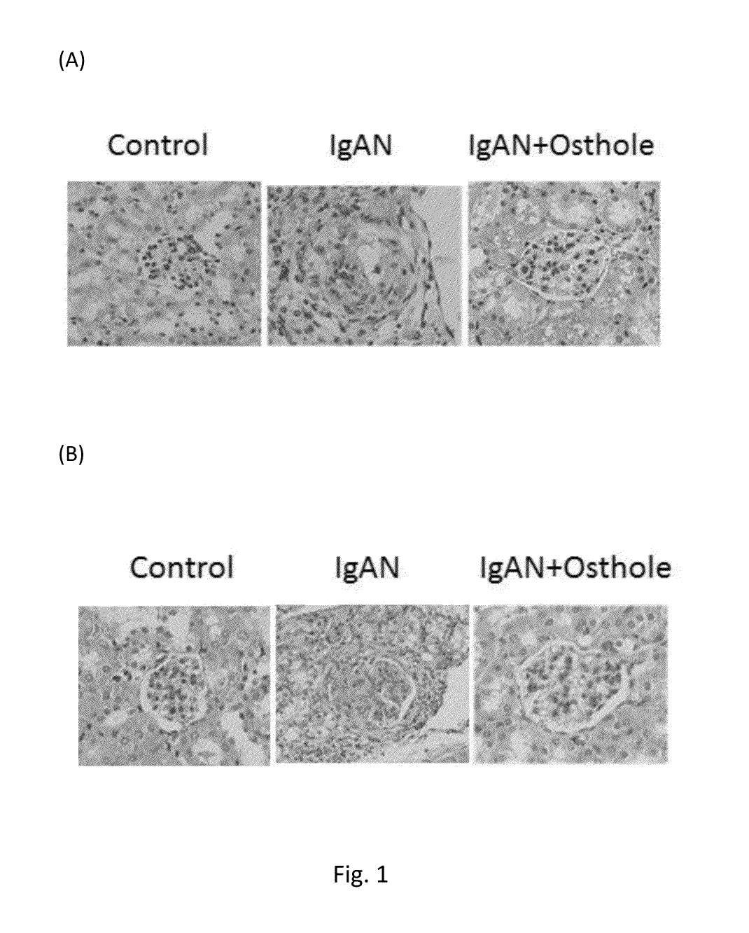

As shown in FIG. 1A, compared to normal control mice, disease-control FSGS mice showed significantly increased renal tissue damages, including proliferation of mesangial cells, disappearance of glomerular capillaries, death of visceral epithelial cells and parietal cells, deposit of fibrous materials and sclerosis, protein casts found in the interstitium and renal tubule atrophy. In contrast, in the osthole treatment mice, the renal tissue damages were significantly reduced. Therefore, the FSGS animal model established herein can be used in subsequent analytical tests, wherein animals receiving osthole can be used as the medical treatment group (representing animals whose symptoms are improved after medical treatment).

2.2 Establishment of IgAN Model

Prg-IgAN was induced in B-cell-deficient (BCD) mice by daily injection of purified IgA anti-phosphorylcholine and pneumococcal C-polysaccharide (PnC) as described previously (Kidney Int 2006; 70: 283-297).

As shown in FIG. 1B, compared to normal control mice, disease-control IgAN mice showed significantly increased renal tissue damages, including glomerular diffuse mesangial proliferation, formation of crescents and sclerosis, inflammation in the renal tubular interstitium (especially around the glomeruli), and renal tubule atrophy accompanied by proteinaceous casts and occasional erythrocyte casts. In contrast, in the osthole treatment mice, the renal tissue damages were significantly reduced. Therefore, the IgAN animal model established herein can be used in subsequent analytical tests, wherein animals receiving osthole can be used as the medical treatment group (representing animals whose symptoms are improved after medical treatment).

2.3 Urine Test

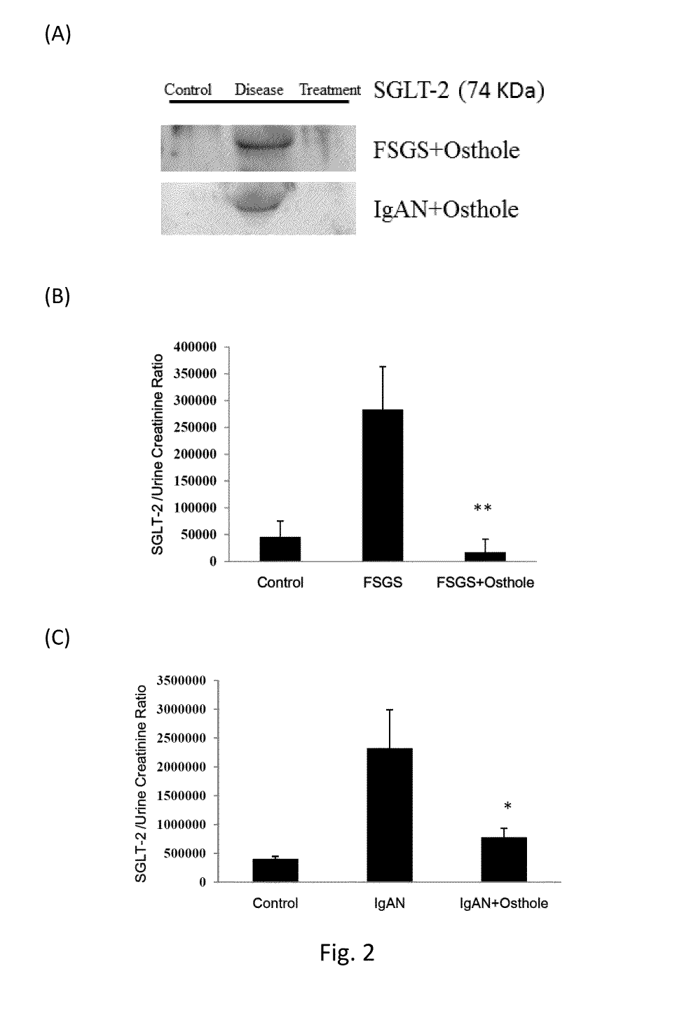

The mouse urine was collected for analysis for SGLT2 expression levels by Western blotting, calibrated with urine creatinine concentration. The results were shown in mean±SEM. Comparison between two groups was conducted using ANOVA or Student's t test. A p value smaller than 0.05 was considered as statistically significant. FIG. 2(A) shows the results of Western blotting. FIG. 2(B) and Fig. (C) show the results after calibration with urine creatinine concentration. SGLT2 expression levels were expressed in percentage as shown in Table 3.

### Table 3: SGLT2 Expression Levels in Animal Urine

TABLE 3
SGLT2 expression levels in animal urine

| | | SGLT2 | |
|---|---|---|---|
| Animal | | Expression | Statistical |
| Experiment Model | Group | Percentage | Challenge |
| FSGS | Normal Control | 6% | P < 0.01 |
| | Disease Control | 100% | |
| | Osthole Treatment | 14% | P < 0.01 |
| IgAN | Normal Control | 7% | P < 0.01 |
| | Disease Control | 100% | |
| | Osthole Treatment | 10% | P < 0.05 |

Biomarker Protein Expression Percentage: (Osthole Treatment Group/Disease Control Group) × 100%; this experiment was conducted individually for 3 times and the means were compared; statistical challenges were passed.

As shown in FIGS. 2(A), (B) and (C), compared with the normal control group, higher SGLT2 protein expression levels were detected in urine samples from the disease control groups of FSGS and IgAN mice. These results have statistical significance and demonstrate that SGLT2 can be used as a marker for FSGS and IgAN. When an individual's urine sample is tested to have higher SGLT2 expression level, it means that said individual has a probability of suffering from a chronic kidney disease, especially FSGS or IgAN.

In addition, as shown in FIGS. 2(A), (B) and (C), after treatment by osthole, the SGLT2 protein expression levels in mice with induced FSGS and IgAN are significantly lower than those in the animals of the disease control group. It suggests that SGLT2 may be used as a biomarker for monitoring the disease status, especially following treatment of the disease. When the SGLT2 expression level in the urine sample of a patient is measured as lower than that before treatment, it means that the disease status of said patient has been improved after treatment.

The above results show that in FSGS and IgAN animal models, the SGLT2 expression level of the disease control group is significantly higher when compared with the normal control group, and thus SGLT2 expression in urine may be used as a diagnostic marker for FSGS or IgAN. In addition, pathological symptoms of the animals in the disease control group are improved after treatment by administering osthole, accompanied by the observation that the protein expression of SGLT2 in urine decreases significantly. This suggests that SGLT2 may be used as a biomarker for monitoring the disease status after treatment of the disease. The present invention may be developed into testing kits for chronic kidney diseases, especially FSGS or IgAN, wherein SGLT2 can be used as a biomarker for screening for chronic kidney diseases and monitoring disease status to assay the efficacy of treatment. The test may be easily accomplished by measuring the proteins in a patient's urine. Without any invasive tests, acceptance among patients will be increased to facilitate early diagnosis and continuous monitoring of the disease status.

What is claimed is:

1. A method for diagnosing a chronic kidney disease in a subject, comprising
(a) obtaining a urine sample from the subject;
(b) determining a level of a sodium-glucose linked transporter 2 (SGLT2) protein in the urine sample; and
(c) identifying the subject as having or at risk for the chronic kidney disease, if the level of the SGLT2 protein in the urine sample is increased relative to the level of the SGLT2 protein in urine from a control.

2. The method of claim 1, wherein the chronic kidney disease is focal segmental glomerulosclerosis (FSGS) or IgA nephropathy (IgAN).

3. The method of claim 1, wherein the urine sample is obtained in a non-invasive way.

4. The method of claim 1, wherein the level of the SGLT2 protein is measured by an immunoassay.

5. The method of claim 1, further comprising applying a further diagnostic test to the subject to confirm occurrence or status of the disease, when the subject is identified as having or at risk for the chronic kidney disease.

6. The method of claim 5, further comprising subjecting the subject to a medical therapy for treating the chronic kidney disease.

7. The method of claim 6, wherein the medical therapy involves administration of a drug for treating the chronic kidney disease, selected from the group consisting of orticosteroid drugs, non-steriodal anti-inflammatory drugs (NSAIDs), immunosuppressants, cytotoxic drugs and vasodilators.

8. A method for monitoring progression of a chronic kidney disease (CKD), comprising
(a) providing a first urine sample from a patient with CKD at a first time point;
(b) providing a second urine sample from the patient at a second time point; which is later than the first time point;
(c) measuring levels of a sodium-glucose linked transporter 2 (SGLT2) protein in the first and second urine samples by an immunoassay; and
(d) determining progression of the chronic kidney disease in the patient based on the levels of the SGLT2 protein in the first and second urine samples, wherein an increased level of the SGLT2 protein in the second urine sample relative to that in the first urine sample is indicative of progression of the chronic kidney disease in the patient.

9. The method of claim 8, wherein the patient is subjected to a treatment for treating the chronic kidney disease, and the first and second biological samples are obtained either before or after the treatment or during the course of the treatment.

10. The method of claim 9, further comprising evaluating efficacy of the treatment for treating the chronic kidney disease, wherein a decrease of the level of SGLT2 protein after the treatment or over the course of the treatment indicates that the treatment is effective on the patient.

11. The method of claim 8, wherein the chronic kidney disease is focal segmental glomerulosclerosis (FSGS) or IgA nephropathy (IgAN).

12. The method of claim 8, wherein the urine is obtained in a non-invasive way.

13. The method of claim 1, wherein the level of the SGLT2 protein is measured by an immune assay.
